(19) United States
(12) Patent Application Publication
(10) Pub. No.: US 2015/0246043 A1
(43) Pub. Date: Sep. 3, 2015

(54) ORAL DOSAGE FORMS FOR MODIFIED RELEASE COMPRISING RUXOLITINIB

(71) Applicant: ratiopharm GmbH, Ulm (DE)

(72) Inventors: Ralph Stefan, Ebenweiler (DE); Frank Sievert, Allmendingen (DE); Katrin Rimkus, Pullach (DE)

(21) Appl. No.: 14/417,226

(22) PCT Filed: Jul. 25, 2013
(86) PCT No.: PCT/EP2013/065771

§ 371 Date: Jan. 26, 2015

(30) Foreign Application Priority Data
Jul. 27, 2012 (EP) 12005485.3

(51) Int. Cl. A61K 31/519; A61J 3/10; A61J 3/00; A61K 9/28

(52) U.S. Cl. CPC A61K 31/519; A61K 9/28; A61K 9/2866; A61J 3/10; A61J 3/005

(57) ABSTRACT

The invention essentially relates to oral dosage forms comprising a JAK kinase inhibitor, preferably Ruxolitinib, suitable for modified release, and processes of preparing such oral dosage forms.

The invention essentially relates to oral dosage forms comprising a pharmaceutically active substance, preferably (3R)-3-cyclopentyl-3-[4-(7H-pyrrolo[2,3-d]pyrimidin-4-yl)pyrazol-1-yl]propanenitrile, its pharmaceutically acceptable salts, its metabolites or pharmaceutically acceptable salts thereof, suitable for modified release, and processes of preparing such oral dosage forms.

(3R)-3-cyclopentyl-3-[4-(7H-pyrrolo[2,3-d]pyrimidin-4-yl)pyrazol-1-yl]propanenitrile has the chemical formula C₁₇H₁₈N₆ and is reported in WO2007070514 as an inhibitor of protein kinases, such as the enzyme Janus Kinase in its subtypes 1 and 2 (hereinafter also referred to as “JAK1” and “JAK2”) and as such it has been asserted that it is useful in the therapy of various diseases, e.g. cancer. The compound is also known under the INN Ruxolitinib and apparently has the following structure (I):

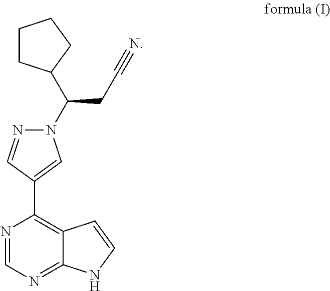 [SMILES: N#CC[C@H](C1CCCC1)n1cc(-c2ncnc3[nH]ccc23)cn1]

Pharmaceutically acceptable salts of Ruxolitinib have been reported in WO 2008/157208 and compounds described as metabolites of Ruxolitinib have been reported in WO 2008/157207 as well as WO 2011/044481.

Ruxolitinib and especially its pharmaceutically acceptable salts like the phosphate salt described in WO 2008/157208 are reported to possess a very high aqueous solubility and belong to Biopharmaceutics Classification System class I (high permeability, high solubility). These properties as well as various other physiological factors such as gastrointestinal pH, enzyme activities, gastric and intestinal transit rates negatively influence the bioavailability of Ruxolitinib and make it difficult to prepare a modified-release formulation of Ruxolitinib or pharmaceutically acceptable salts of Ruxolitinib as well as the described metabolites of Ruxolitinib or pharmaceutically acceptable salts thereof.

Hence, there is a need for the provision of pharmaceutical dosage forms and processes for the manufacture of these pharmaceutical dosage forms comprising Ruxolitinib, which do not suffer from the above mentioned draw-backs. Preferably, an oral dosage form should be provided having improved properties like content-uniformity, solubility, dissolution profile, well-defined, predictable and reproducible dissolution rates, stability, and bioavailability. Such an oral dosage form should be producible in a large scale in an economic beneficial way and should be able to provide desirable plasma levels of the drug.

The inventors of the present invention unexpectedly have found that the above drawbacks can be overcome by providing oral dosage forms for modified release comprising
(a) Ruxolitinib, and
(b) a non-erodible material.

It is found that the dosage forms of the present invention have the advantage that the Ruxolitinib is gradually released over a relatively long period so that the drug is maintained in the blood stream for a superior long time and at a superior uniform concentration. This allows administration e.g. only once daily. Administration of the oral dosage forms of the present invention results in superior blood level fluctuation, that means periods of transient therapeutic overdose, followed by a period of therapeutic underdosing can be avoided. Consequently, the dosage forms of the present invention, particularly provide a constant release of Ruxolitinib, preferably over a prolonged period of time, which avoids blood level fluctuations of the drug in the patient.

Moreover, the dosage form of the present invention is released in the gastrointestinal tract of the patient but not in the stomach, in order to avoid a “nervous stomach” or nausea.

A further subject of the present invention is a process for manufacturing the oral dosage forms of the present invention, preferably in form of a modified release tablet or capsule.

In the following, explanations regarding the pharmaceutical dosage form of the present invention are given. However, these explanations also apply to the processes for manufacturing the pharmaceutical dosage form, such as the modified release tablet or capsule of the present invention, and to the use of the present invention.

Within the present application generally the term “modified release” is used as defined by the USP. Preferably, modified release dosage forms are those whose drug release characteristics accomplish therapeutic or convenience objectives not offered by immediate release forms. Generally, immediate release (IR) forms release at least 70% of the drug within 1 hour or less. The term “modified release” can comprise delayed release, prolonged release, sustained release, extended release and/or controlled release.

Delayed release usually indicates that the drug (=Ruxolitinib) is not being released immediately after administration but at a later time, preferably less than 10% are released within two hours after administration.

Prolonged release usually indicates that the drug (=Ruxolitinib) is provided for absorption over a longer period of time than IR forms, preferably for about 2 to 24 hours, in particular for 3 to 12 hours.

Sustained release usually indicates an initial release of drug (=Ruxolitinib), sufficient to provide a therapeutic dose soon after administration, preferably within two hours after administration, and then a gradual release after an extended period of time, preferably for about 3 to 18 hours, in particular for 4 to 8 hours.

Extended release usually indicates a slow drug (=Ruxolitinib) release, so that plasma concentrations are maintained at a therapeutic level for a time period of between 6 and 36 hours, preferably between 8 and 24 hours.

Controlled release dosage forms usually release the drug (=Ruxolitinib) at a constant rate and provide plasma concentrations that remain essentially invariant with time.

In a preferred embodiment the oral dosage form of the present invention is an extended release dosage form.

In particular, the oral dosage form of the present invention shows a drug release of less than 10% within 2.0 hours. Further, the oral dosage form of the present invention shows a drug release of more than 80% within 3.0 to 12.0 hours, preferably between 4.0 and 8.0 hours.

Generally, within this application the release profile is determined according to USP 31-NF26 release method, apparatus II (paddle). The measurements are carried out in 0.1 HCl at 37° C., wherein the stirring speed is 75 rpm, and re-buffering after 2 hours to pH 6.8.

In a preferred embodiment, the oral dosage form of the present invention is a solid oral dosage form, in particular a solid peroral dosage form.

The term Ruxolitinib (component (a)) as used in the present invention relates to the compound as shown in formula I (free base) or to pharmaceutically acceptable salts thereof, preferably pharmaceutically acceptable acid addition salts, e.g. as described in WO 2008/157208. The acids, which are used to prepare the pharmaceutically acceptable acid addition salts, are preferably those which form non-toxic acid addition salts.

In the oral dosage form of the present invention, Ruxolitinib as the active ingredient (component (a)) can be provided in amorphous form, in crystalline form or as a mixture of both forms. In a preferred embodiment of the present invention Ruxolitinib is provided as the phosphate. Most preferred is the crystalline form of the phosphate of Ruxolitinib.

In a preferred embodiment the oral dosage form of the present invention comprises 1.0 to 60 wt. %, more preferably 2.0 to 30 wt.-%, still more preferably 3.0 to 20 wt. %, in particular 4.0 to 15 wt. % Ruxolitinib, based upon the total weight of the oral dosage form and based on the weight of Ruxolitinib in form of the free base, i.e. as shown in formula (I) above.

In a preferred embodiment the oral dosage form of the present invention comprises 1.0 to 100 mg, more preferably 10 to 75 mg, in particular 25 to 50 mg, based on Ruxolitinib in form of the free base, i.e. as shown in formula (I) above.

The modified release tablet of the present invention further contains a non-erodible material (b). Generally, the non-erodible material is suitable as release controlling agent.

In a first embodiment the non-erodible material can be described as providing a scaffold (matrix) for embedding the active ingredient and to form a physical barrier, which hinders the active ingredient from being released immediately from the dosage form. Thus, the non-erodible material has the effect that the active ingredient can be released from the scaffold in continuous manner. Release of the drug from the matrix can further be by dissolution controlled as well as diffusion controlled mechanisms. In this first embodiment the non-erodible material functions as matrix forming material.

In a second embodiment the non-erodible material can be described as a shell-forming material. Preferably, in that embodiment the oral dosage form is a tablet. The release modifying shell preferably encompasses the drug containing tablet core.

In a third embodiment the non-erodible material can be described as release modifying coating in a multiple unit pellet system (MUPS).

Generally, (i.e. for all three above described embodiments) the oral dosage form of the present invention further comprises a non-erodible material (b). Non-erodible materials are materials, which are able to provide modified release properties, preferably due to their limited solubility, more preferably due to their limited solubility in aqueous conditions at pH 5.0. Preferably, the non-erodible polymer has a water solubility of less than 33 mg/I at a temperature of 25° C. at a pH of 5.0, more preferably of less than 22 mg/I, still more preferably of less than 11 mg/I, especially from 0.01 to 5 mg/I. The water-solubility is determined according to the column elution method of the Dangerous Substances Directive (67/548/EEC), Annex V, Chapter A6. The pH value is determined according to Ph. Eur. 6.0, 2.2.3. The pH value of the aqueous medium usually is achieved by addition of HCl (or NaOH), if necessary.

The solubility of the non-erodible material can be pH independent or pH dependent. Both embodiments are preferred. If the non-erodible material is pH dependent, it is preferred that the non-erodible material has a solubility in water at 25° C. at a pH of 7.0 of more than 33 g/l, more preferably of 50 g/l to 10,000 g/l, still more preferably from 100 g/l to 5,000 g/l, in particular from 200 g/l to 2,000 g/l.

The non-erodible material can comprise an inert non-erodible material, a lipid non-erodible material and/or a hydrophilic non-erodible material. Examples for an inert non-erodible material are ethylcellulose, methacrylate copolymer, polyamide, polyethylene, and polyvinyl acetate; examples for lipid non-erodible materials are carnauba wax, cetyl alcohol, hydrogenated vegetable oils, microcrystalline waxes, monoglycerides, triglycerides and PEG monostearate; examples for hydrophilic non-erodible materials are alginates, carbopol, gelatin, hydroxypropyl cellulose, hydroxypropyl methylcellulose, methylcellulose, xanthan gum and polyethylene oxide.

In a preferred embodiment the non-erodible material is a non-erodible polymer. The non-erodible polymer usually has a weight average molecular weight ranging from 30.000 to 3,000,000 g/mol, preferably from more than 50,000 to 2,500,000 g/mol, more preferably from more than 125,000 to 2,000,000 g/mol, still more preferably from 250,000 to 2,200,000 g/mol, particularly preferred from 400,000 to 1,500,000 g/mol. Furthermore, a 2% w/w solution of the non-erodible polymer in water at pH 7.0 preferably has a viscosity of more than 2 mPas, more preferably of more than 5 mPas, particularly more than 8 mPas and up to 850 mPas, when measured at 25° C. The viscosity is determined according to Ph. Eur., 6^{th} edition, Chapter 2.2.10. In the above definition the term “solution” may also refer to a partial solution (in case that the polymer does not dissolve completely in the solution). The weight average molecular weight is preferably determined by gel electrophoresis.

It is further preferred that the non-erodible polymer has a melting temperature of below 220° C., more preferably of between 25° C. and 200° C. In a particularly preferred embodiment the melting temperature is between 35° C. and 190° C. The determination of the melting temperature is carried out according to Ph. Eur., 6^{th} edition, Chapter 2.2.15.

If the non-erodible material b) is a polymeric material, it preferably can be selected from acrylic polymers or acrylic copolymers such as polymers obtained from acrylic acid and/or methacrylic acid monomers. Other preferred polymers include, but are not limited to, cellulose and cellulose derivatives such as cellulose acetate phthalate (CAP), hydroxypropyl methyl cellulose (HPMC), hydroxypropyl cellulose (HPC), hydroxypropyl methyl cellulose acetate (HPMCA), hydroxypropyl methyl cellulose phthalate (HPMCP) and cellulose acetate succinate (CAS), polyvinyl polymers such as polyvinyl alcohol phthalate, polyvinyl acetate phthalate and polyvinyl butyl phthalate, and mixtures of one or more of these polymers.

In particular, the following kinds of non-erodible polymers are particularly preferred.

1. Cellulose ether, such as ethyl cellulose, hydroxypropyl methyl cellulose (HPMC) and hydroxypropyl cellulose (HPC), preferably ethyl cellulose, preferably ethyl cellulose having an average molecular weight of 150,000 to 300,000 g/mol and/or an average degree of substitution, ranging from 1.8 to 3.0, preferably from 2.2 to 2.6. This embodiment preferably is used for MUPS or core/shell-tablets;
2. Cellulose ester, preferably cellulose acetate phthalate, carboxymethyl ethyl cellulose, hydroxypropyl methylcellulose phthalate. This embodiment is preferably used for core/shell tablets;
3. Copolymers of methacrylic acid or methacrylic acid esters, preferably ethylacrylate-methyl methacrylate-trimethylammonioethyl methacrylate-chloride ethylacrylate-methyl-methacrylate-trimethylammonioethylmethacrylate-chloride ethylacrylate-methylmeth-acrylate, methacrylic acidmethylmethacrylate, wherein the weight ratio is 1:2 methacrylic acid-methylmethacrylate, wherein the weight ratio is 1:1; preferred acrylic polymers are e.g. polyacrylate, polymethacrylate as well as derivatives and mixtures or copolymers thereof;
4. Polyvinyl acetate or polyvinyl acetate copolymers, preferably polyvinyl acetate phthalate; and mixtures thereof.

The non-erodible material (b) is contained in the tablet or capsule in an amount of 5 to 80 wt. %, preferably from 10 to 50 wt. %, most preferably from 15 to 40 wt. %, based upon the total weight of the oral dosage form. If too little non-erodible material is used, the formulations may break up during the passage down the gastrointestinal tract and this, in turn, may lead to a premature release of a large portion of the content of the drug. If too much matrix former is used, there is a risk that some of the drug will be encapsulated and not released from the tablet.

The oral dosage form of the invention further optionally comprises a pore-forming material c). The term “channeling agent” is in the art often synonymously used for the pore-forming material of the present invention. Since the pore-forming material is generally soluble in the gastrointestinal tract and leaches out from the oral dosage form, the pore-forming material can be described has having the effect of forming pores, such as small holes within the tablet, through which the active ingredient can be released from the tablet matrix in a controlled manner. Thus, release of the active ingredient generally depends on dissolving the pore forming material and thereby forming a porous matrix of capillaries such that the drug can leach out of the matrix.

The pore-forming substance usually has a water solubility of more than 50 mg/I, preferably more than 100 mg/I, at a temperature of 25° C. and pH 5.0, more preferred of more than 250 mg/I and particularly preferred of more than 25 g/l. The water-solubility of the pore-forming substance may range up to 2.5 kg/l. The water-solubility is determined according to the column elution method of the Dangerous Substances Directive (67/548/EEC), Annex V, Chapter A6.

The pore-forming substances can be selected from inorganic substances, preferably from inorganic salts such as NaCl, KCl, Na₂SO₄. Furthermore, the pore-forming substances can be selected from organic substances, in particular from organic substances being solid at 30° C. and having the above-mentioned water solubility. Suitable examples are PEG, particularly PEG, having a weight average molecular weight of from 2,000 to 10,000 g/mol.

Furthermore, polyvinylpyrrolidone, preferably having a weight average molecular weight of from 5,000 to 29,000 g/mol, PEG with a weight average molecular weight of 380-4800, polyethylene oxide with a weight average molecular weight of less than 100,000 and a viscosity of less than 20 mPa·s, sugar alcohols like mannitol, sorbitol, xylitol, isomalt, and mono or disaccharides, like lactose, are also suitable as pore-forming substances.

The pore forming material is usually contained in the tablet in an amount of 1 to 50 wt. %, preferably from 2 to 40 wt. %, most preferably from 5 to 30 wt. %, based upon the total weight of the oral dosage form.

The tablet of the present invention can further comprise at least one excipient (d) selected from fillers (d1), disintegrants (d2), lubricants (d3), surfactants (d4), glidants (d5), anti-sticking agents (d6), plasticizers (d7) and mixtures thereof.

Generally, fillers are used to top up the volume for an appropriate oral deliverable dose, when low concentrations of the active pharmaceutical ingredients (about 30 wt. % or lower) are present. Preferred fillers of the invention are calcium phosphate, saccharose, calcium carbonate, calcium silicate, magnesium carbonate, magnesium oxide, maltodextrin, glucopyranosyl mannitol, calcium sulfate, dextrate, dextrin, dextrose, hydrogenated vegetable oil and/or cellulose derivatives, such as microcrystalline cellulose. A pharmaceutical composition according to the invention may comprise an inorganic salt as a filler. Preferably, this inorganic salt is dicalcium phosphate, preferably in form of the dihydrate (dicafos).

Dicalcium phosphate dihydrate is insoluble in water, non-hygroscopic, but still hydrophilic. Surprisingly, this behavior contributes to a high storage stability of the composition.

Usually, fillers can be used in an amount of 0 to 60 wt. %, preferably of 5 to 40 wt. %, based on the total weight of the composition.

The oral composition of the present invention can further comprise one or more of a disintegrant. In a preferred embodiment of the invention, the tablet does not contain a disintegrant.

Generally, disintegrants are compounds, capable of promoting the break up of a solid composition into smaller pieces when the composition gets in contact with a liquid, preferably water.

Preferred disintegrants are sodium carboxymethyl starch, cross-linked polyvinylpyrrolidone (crospovidone), sodium carboxymethyl glycolate (e.g. Explotab®), swelling polysaccharide, e.g. soya polysaccharide, carrageenan, agar, pectin, starch and derivates thereof, protein, e.g. formaldehyde—casein, sodium bicarbonate or mixtures thereof. Crospovidone is particularly preferred as disintegrant. Furthermore, a combination of crospovidone and agar is particularly preferred.

Usually, disintegrants can be used in an amount of 0 to 20 wt. %, preferably of 1 to 10 wt. %, based on the total weight of the composition.

In a preferred embodiment of the present invention the oral dosage form is free of any disintegrants.

The oral dosage form of the present invention might further comprise one or more of a surfactant. Preferably, sodium lauryl sulfate is used as surfactant.

Usually, surfactants can be used in an amount of 0.05 to 2 wt. %, preferably of 0.1 to 1.5 wt. %, based on the total weight of the oral dosage form.

Additionally, the oral dosage form of the present invention may comprise a lubricant, a glidant and/or an anti-sticking agent.

In a preferred embodiment of this invention, a lubricant may be used. Lubricants are generally employed to reduce dynamic friction. The lubricant preferably is a stearate, talcum powder or fatty acid, more preferably, hexanedioic acid or an earth alkali metal stearate, such as magnesium stearate. The lubricant is suitably present in an amount of 0.1 to 3 wt. %, preferably about 0.5 to 1.5 wt. % of the total weight of the composition. Preferably, the lubricant is applied in a final lubrication step during the powder preparation. The lubricant generally increases the powder flowability.

The glidant can for example be colloidal silicone dioxide (e.g. Aerosil®). Preferably, the glidant agent is present in an amount of 0 to 8 wt. %, more preferably at 0.1 to 3 wt. % of the total weight of the composition. Preferably, the silicone dioxide has a specific surface area of 50 to 400 m²/g, measured by gas adsorption according to Ph. Eur., 6th edition, Chapter 2.9.26. multipoint method, volumetric determination

The anti-sticking agent is for example talcum and may be present in amounts of 0.05 to 5 wt. %, more preferably in an amount of 0.5 to 3 wt. % of the total weight of the composition.

Furthermore, in a preferred embodiment the pharmaceutical composition of the present invention further comprises one or more plasticizers. The “plasticizers” usually are compounds capable of lowering the glass transition temperature (T_{g}) of the non-erodible material, preferably the non-erodible polymer, preferably of lowering T_{g} from 1 to 50° C., especially from 5 to 30° C. Plasticizers usually are low molecular weight compounds (having a molecular weight from 50 to 500 g/mol) and comprise at least one hydrophilic group.

Examples of suitable plasticizers are dibutyl sebacetate (DBS), Myvacet® (acetylated monoglycerides), triacetin (GTA), citric acid esters, like acetyltriethyl citrate (ATEC) or triethyl citrate (TEC), propylene glycol, dibutyl phthalate, diethyl phthalate, or mixtures thereof.

The combined use of the non-erodible polymer (b) and the pore-forming substance (c) and optionally the plasticizer preferably is capable of modifying the drug release rate. The use of plasticizers is particularly preferred in the third embodiment concerning MUPS.

Regarding the above mentioned pharmaceutically acceptable excipients, the application generally refers to “Lexikon der Hilfsstoffe für Pharmazie, Kosmetik and angrenzende Gebiete”, edited by H. P. Fiedler, 5^{th} Edition, Editio Cantor Verlag, Aulendorf and earlier editions, and “Handbook of Pharmaceutical Excipients”, third edition, edited by Arthur H. Kibbe, American Pharmaceutical Association, Ishington, USA, and Pharmaceutical Press, London.

In the tablet according to the present invention the non-erodible material (b), the pore forming material (c) and/or the at least one excipient (d) preferably have a surface of 0.2 to 10 m²/g, preferably of 0.3 to 8 m²/g, most preferably of 0.4 to 5 m²/g, as measured by gas adsorption according to Ph. Eur., 6th edition, Chapter 2.9.26, multipoint method, volumetric determination.

In the tablet of the invention the at least one non-erodible material (b), the pore forming material (c) and/or the excipient(s) (d) generally show a plastic behavior, such as a ductile behaviour. This behavior can be described by the yield pressure of the material. The materials of components (a), (b) and/or (c) generally have a yield pressure of less than 150 MPa, preferably less then 100 MPa, most preferably of less than 75 MPa. If the yield pressure is above 150 MPa, the material is too brittle and causes difficulties in being compressed into a tablet, bearing the risk that the tablet breakes or crumbles. The yield pressure can be determined from a Heckel plot. According to Heckel, there is a linear relationship between the relative porosity (inverse density) of a powder and the applied pressure. The slope of the linear regression is the Heckel constant, a material dependent parameter inversely proportional to the mean yield pressure (the minimum pressure required to cause deformation of the material undergoing compression). Thus, the yield pressure is obtained by measuring the reciprocal value from the slope of the Heckel plot.

In this context it is generally noted that, due to the nature of pharmaceutical excipients, it cannot be excluded that a certain compound meets the functional requirements of more than one of the above mentioned excipient classes. However, in order to enable an unambiguous distinction and terminology in the present application, the same pharmaceutical compound can only be subsumed as one of the functional excipient classes presented above. For example, if microcrystalline cellulose is used as a filler, it cannot additionally classify as a disintegrant (although microcrystalline cellulose has some disintegrating properties).

As explained above, the present invention concerns three preferred embodiments of the solid oral dosage form. Hence, the present invention further relates to three preferred embodiments of a process for producing said oral dosage forms.

In the first preferred embodiment the present invention concerns a matrix dosage form, preferably a matrix tablet. The matrix tablet preferably is produced by a process 10, comprising the steps of
(1-I) providing (and optionally blending) components (a), (b), optionally c), and optionally (d),
(1-II) optionally agglomerating the components of step (I) to yield granules,
(1-III) compressing the mixture resulting from step (I) or (II) into tablets; and
(1-IV) optionally coating the tablets, preferably with a suitable film (e).

In this first preferred embodiment of the invention, the dosage form preferably comprises Ruxolitinib, a non-erodible material, a pore-forming material, a filler, a glidant and a lubricant. In a further preferred embodiment, the composition comprises from 5 to 20 wt. % of Ruxolitinib, from 15 to 60 wt. % of non-erodible material, from 10 to 40 wt. % of a pore-forming material, from 10 to 40 wt % of a filler, from 1 to 10 wt. % of a glidant and from 1 to 10 wt. % of a lubricant, based upon the total weight of the dosage form. In an alternative preferred embodiment, the non-erodible material is present in an amount of from 25-60 wt %.

In a second preferred embodiment of the invention, the oral dosage form is in form of a tablet, comprising a core and a shell, wherein the core comprises components (a) and optionally (c) and/or (d), and wherein the shell comprises components (b) and optionally (c) and/or (d).

Process for manufacturing a tablet according to any of the claim 1 to 9 or 11 comprising the steps of
(2-I) mixing components (a) and optionally (c) and/or (d),
(2-II) optionally agglomerating the components of step (I) to yield granules,
(2-III) compressing the mixture into tablets, and
(2-IV) coating the tablets with a coating comprising components (b) and optionally (c) and/or (d).
(2-V) Optionally, the resulting tablets can be film-coated with a suitable film (e).

The preferred processes of the first and second embodiment are described below in more detail.

In step (1-I) or (2-I) components (a), (b), (c) and/or (d) can be provided in micronized form. Micronization can be carried out by milling, such as in a air jet mill. Preferably, the mean particle size (D50) of Ruxolitinib (a) is from 20 to 120 μm, and from components (b), (c) and/or (d) it is from 30 to 150 μm.

Optionally, the ingredients of the tablet of the invention are blended in order to provide a formulation having a homogenous distribution of Ruxolitinib (a) within the formulation.

Blending can be carried out with conventional mixing devices, e.g. in a free-fall mixer like Turbula® T10B (Bachofen AG, Switzerland). Blending can be carried out e.g. for 1 minute to 30 minutes, preferably for 2 minutes to less than 10 minutes.

Generally, the step (1-II) or (2-II) of “agglomerating” components (a) to (d) (components (c) and (d) optional) refers to a process, wherein particles are attached to each other, thereby giving larger particles. The attachments may occur through physical forces, preferably van der Waals forces. The attachment of particles preferably does not occur through chemical reactions.

Agglomeration (II) can be carried out in different devices. For example, agglomeration can be carried out by a granulation device, preferably by a dry granulation device. More preferably, agglomeration can be carried out by intensive blending. For example, agglomeration can be carried out by blending in a free-fall mixer or a container mixer. An example for a suitable free fall mixer is Turbula® T10B (Bachofen AG, Switzerland). Generally, the blending is carried out for a time, being long enough for agglomeration to occur. Usually, blending is carried out for 10 minutes to 2 hours, preferably for 15 minutes to 60 minutes, more preferably from 20 minutes to 45 minutes.

In a preferred embodiment the agglomeration step is carried out as a dry-compaction step. In a preferred embodiment the dry-compaction step is carried out by roller compaction. Alternatively, e.g. slugging can be used. If roller compaction is applied, the compaction force usually ranges from 1 to 30 kN/cm, preferably from 2 to 20 kN/cm, more preferably from 2 to 10 kN/cm. The gap width of the roller compactor usually is 0.8 to 5 mm, preferably 1 to 4 mm, more preferably 1.5 to 3.2 mm, especially 1.8 to 3.0 mm. After the compaction step, the received comprimate preferably is granulated. Preferably, the granulation step is carried out by an elevated sieving equipment, e.g. Comil® U5 (Quadro Engineering, USA). Alternatively, compaction and granulation can be carried out within one device.

That means, the agglomeration step preferably is carried out in the absence of solvents, preferably in the absence of organic solvents and/or in the absence of water and preferably results in agglomerates or granules.

In a preferred embodiment the agglomeration conditions in step (1-II) or (2-II) are chosen such that the resulting agglomerated pharmaceutical composition comprises a volume mean particle size (D50) of 5 to 500 μm, more preferably of 20 to 250 μm, further more preferably of 50 to 200 μm.

The bulk density of the agglomerated pharmaceutical composition made by the process of the present invention generally ranges from of 0.1 to 0.85 g/ml, preferably of from 0.25 to 0.85 g/ml, more preferably of from 0.3 to 0.75 g/ml.

In a preferred embodiment the composition has a bulk density of 0.5 to 0.8 g/ml when used for direct compressing and 0.1 to 0.5 when used for dry compaction.

The Hausner factor of the agglomerated (or granulated) composition is less than 1.3, preferably less than 1.2 and most preferably less than 1.15. The agglomerated pharmaceutical composition resulting from step (iii) of the invention preferably possesses Hausner ratios in the range of 1.02 to 1.5, preferably of 1.05 to 1.4, more preferably between 1.08 to 1.3. The Hausner ratio is the ratio of tapped density to bulk density. Bulk density and tapped density are determined according to USP 24, Test 616 “Bulk Density and Tapped Density”.

The compression step (1-III) or (2-III), can be carried out on a rotary press, e.g. on a Fette® 102i (Fette GmbH, Germany) or a Riva® piccola (Riva, Argentina). If a rotary press is applied, the main compaction force usually ranges from 1 to 50 kN, preferably from 2 to 40 kN, more preferably from 3.5 to 30 kN. The resulting tablets usually have a hardness of 30 to 100N, preferably of 50 to 85 N.

The shell of the tablets of the second preferred embodiment of the present invention is applied in process step (2-IV). Said step comprises coating the tablet core with a coating comprising preferably components (b) and optionally (c) and/or (d). Preferably, the coating comprises components (b), (c) and a plasticizer.

The coating process is generally carried out in a continuously process in a pan coater or a fluid bed dryer. The coating process is preferably carried out on a pan coater, e.g. on a Lödige LHC 25 (Lödige GmbH, Germany). If a pan coater is applied, the spray pressure usually ranges from 0.8-2 bar, preferably from 1-1.5 bar. The product temperature varies according to the applied polymer. Usually the product temperature is adjusted by 20-40° C., preferably from 32-38° C.

The coating usually has a thickness of 0.01 to 2 mm, preferably from 0.1 to 1.5 mm, more preferably from 0.2 to 1 mm.

After having received the compressed tablets, in both preferred processes the compressed tablet could be film-coated (step 1-IV or 2-V).

In the present invention, the following three types of film-coatings are possible:
e1) film-coating without effecting the release of the active ingredient (preferred),
e2) gastric juice resistant film-coatings,
e3) retard coatings.

Film-coatings without effecting the release of the active ingredient are preferred. Generally, said coating is water-soluble (preferably having a water solubility at 25° C. of more than 250 mg/ml). In gastric juice resistant coatings the solubility depends on the pH of the surrounding. Retard coatings are usually non-soluble (preferably having a water solubility at 25° C. of less than 10 mg/ml).

Generally, film-coatings e1) are prepared using cellulose derivatives, poly(meth)-acrylate, polyvinyl pyrrolidone, polyvinyl acetatephthalate, and/or shellac or natural rubbers such as carrageenan.

Preferred examples of coatings, which do not affect the release of the active ingredient are those including methylcellulose (MC), hydroxypropyl methylcellulose (HPMC), hydroxypropyl cellulose (HPC), hydroxyethyl cellulose (HEC), polyvinyl pyrrolidone (PVP) and mixtures thereof. These polymers generally have a median molecular weight of 10,000 to 150,000 g/mol.

A preferred polymer is HPMC, most preferably a HPMC having a median molecular weight of 10,000 to 150,000 g/mol and a median level of substitution of —OCH₃-residues of 1.2 to 2.

Examples of gastric juice resistant coatings e2) are cellulose acetate phthalate (CAP), hydroxypropyl methylcellulose phthalate (HPMCP and polyvinyl acetate phthalate (PVAP). Examples of retard coatings e3) are ethyl cellulose (EC, commercially available e.g. as Surelease®) and poly(meth)acrylate (commercially available e.g. as Eudragit® RL or RS and L/S).

The coating e) can be free of active ingredient. However, it is also possible that the coating contains active ingredient (Ruxolitinib). In such a case, that amount of active ingredient would function as an initial dose. In such a case the coating e) preferably comprises 1 to 45 wt. %, preferably 5 to 35 wt. %, most preferably 10 to 30 wt. % of Ruxolitinib, based on the total amount of Ruxolitinib contained in the tablet. In this embodiment, the coating preferably is a coating, which does not effect the release of Ruxolitinib.

In case the film coating does not contain Ruxolitinib (which is preferred), it usually has a thickness of 2 μm to 100 μm, preferably from 20 to 60 μm. In case of a coating containing Ruxolitinib, the thickness of the coating is usually 10 μm to 2 mm, more preferably from 50 to 500 μm.

Accordingly, in a further embodiment the subject invention relates to a tablet in which 1 to 45 wt. %, preferably 5 to 35 wt. %, most preferably 10 to 30 wt. % of the total amount of the Ruxolitinib contained in the tablet, are present as initial doses having immediate release, and 55 to 99 wt. %, preferably 65 to 95 wt. %, most preferably 70 to 90 wt. % of the active ingredient are present in the tablet as a modified release formulation.

In a third aspect of the present invention Ruxolitinib may be incorporated into an osmotic controlled release device. Such devices have at least two components: (a) the core which contains an osmotic agent and Ruxolitinib; and (b) a water permeable, non-dissolving coating, which comprises the non-erodible material surrounding the core, the coating controlling the influx of water to the core from an aqueous environment of use so as to cause drug release by extrusion of some or all of the core to the environment of use. The osmotic agent contained in the core of this device may be an aqueous-swellable hydrophilic polymer or it may be an osmogen. The coating is preferably polymeric, aqueous-permeable, and has at least one delivery port. Examples of such devices are disclosed more fully in U.S. Pat. No. 6,706,283 the disclosure of which is hereby incorporated by reference.

In addition to Ruxolitinib, the core comprises a osmotic agent, which creates a driving force for transport of water from the environment of use into the core of the device. Exemplary osmotic agents are water-swellable hydrophilic polymers. The amount of water-swellable hydrophilic polymers present in the core may range from about 5 to about 80 wt %, preferably 10 to 50 wt %. Exemplary materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Typical classes of suitable osmotic agents are water-soluble organic acids, salts and sugars that are capable of imbibing water to thereby effect an osmotic pressure gradient across the barrier of the surrounding coating. Typical useful osmogens include magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, sucrose, glucose, fructose, lactose, and mixtures thereof. The core may include a wide variety of additives and excipients that enhance the performance of the dosage form or that promote stability, tabletting or processing.

Such osmotic delivery devices may be fabricated in various geometries including bilayer, wherein the core comprises a drug layer and a sweller layer adjacent to each other; trilayer, wherein the core comprises a sweller layer “sandwiched” between two drug layers; and concentric, wherein the core comprises a central sweller composition surrounded by the drug layer.

The coating of such a tablet comprises a non-erodible membrane insoluble in water, but permeable to water and substantially impermeable to drug and excipients contained within. The coating contains one or more exit passageways or ports in communication with the drug-containing layer(s) for delivering the drug composition. The drug-containing layer(s) of the core contains the drug composition while the sweller layer consists of an expandable hydrogel, with or without additional osmotic agents. When placed in an aqueous medium, the tablet imbibes water through the membrane, causing the composition to form a dispensable aqueous composition, and causing the hydrogel layer to expand and push against the drug-containing composition, forcing the composition out of the exit passageway. The composition can swell, aiding in forcing the drug out of the passageway. Drug can be delivered from this type of delivery system either dissolved or dispersed in the composition that is expelled from the exit passageway.

In the case of a bilayer geometry, the delivery pots) or exit passageway(s) may be located on the side of the tablet containing the drug composition or may be on both sides of the tablet or even on the edge of the tablet so as to connect both the drug layer and the sweller layer with the exterior of the device. The exit passageway(s) may be produced by mechanical means or by laser drilling, or by creating a difficult-to-coat region on the tablet by use of special tooling during tablet compression or by other means.

A particularly useful embodiment of an osmotic device comprises: (a) a single-layer compressed core comprising: (i) Ruxolitinib (ii) a hydroxyethylcellulose, and (iii) an osmotic agent, wherein the hydroxyethylcellulose is present in the core from about 2.0% to about 35% by weight and the osmotic agent is present from about 15% to about 70% by weight; (b) a water-permeable layer surrounding the core; and (c) at least one passageway within the layer (b) for delivering the drug to a fluid environment surrounding the tablet.

Several disintegrants tend to form gels as they swell with water, thus hindering drug delivery from the device. Non-gelling, non-swelling disintegrants provide a more rapid dispersion of the drug particles within the core as water enters the core. Preferred non-gelling, non-swelling disintegrants are resins, preferably ion-exchange resins. A preferred resin is Amberlite™ IRP 88 (available from Rohm and Haas, Philadelphia, Pa.). When used, the disintegrant is present in amounts ranging from about 1-25% of the core composition.

Another example of an osmotic device is an osmotic capsule. The capsule shell or portion of the capsule shell can be semipermeable.

Coating is conducted in conventional fashion, typically by dissolving or suspending the coating material in a solvent and then coating by dipping, spray coating or preferably by pan-coating. A preferred coating solution contains 5 to 15 wt % polymer. Typical solvents useful with the cellulosic polymers mentioned above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described in U.S. Pat. No. 5,612,059, the pertinent disclosures of which are incorporated herein by reference.

Coatings may also be hydrophobic microporous layers wherein the pores are substantially filled with a gas and are not wetted by the aqueous medium but are permeable to water vapor, as disclosed in U.S. Pat. No. 5,798,119, the pertinent disclosures of which are incorporated herein by reference. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Especially preferred hydrophobic microporous coating materials include polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed in U.S. Pat. Nos. 4,247,498; 4,490,431 and 4,744,906, the disclosures of which are also incorporated herein by reference.

Osmotic control led-release devices may be prepared using procedures known in the pharmaceutical arts. See for example, Remington: The Science and Practice of Pharmacy, 20th Edition, 2000.

The fourth preferred embodiment of the present invention relates to a multiple unit pellet system (MUPS). As the name implies, this type of dosage form comprises more than one discrete unit. Typically, such systems comprise 2 to 50, preferably 3 to 30 discrete units. Typically, such discrete units are coated spheroids. Preferably, such coated spheroids are filled into capsules, preferably hard gelatin capsules. Alternatively, such coated spheroids are compressed into tablets.

Hence, a further subject of the present invention is a process for manufacturing an oral modified release dosage form comprising Ruxolitinib, comprising the steps of
(3-I) providing a pellet core,
(3-II) spraying a solution or suspension comprising component (a) and optionally (d) onto the pellet core,
(3-III) spraying a solution or suspension comprising component (b) and optionally (c) and/or (d) onto the pellet, preferably onto the pellet resulting from step (3-II),
(3-IV) optionally blending the pellets with components (b) and (c) and/or (d); and
(3-V) further processing the resulting mixture into a final oral dosage form.

In this pellet layering embodiment, the present invention provides a process for the manufacture of a modified release dosage form comprising Ruxolitinib, employing a pellet layering process.

In step (3-I) a pellet core is provided. Preferably, the pellet core is a so-called neutral pellet core, that means it does not comprise an active ingredient. Such pellet cores are known in the art as nonpareils. The pellet core can be made of suitable materials, e.g. cellulose, sucrose, starch or mannitol or combinations thereof.

Suitable pellet cores are commercially available under the trade name Cellets® and preferably comprise a mixture of lactose and microcrystalline cellulose.

Furthermore, in a preferred embodiment, pellet cores commercially available as Suglets® are used. Those preferred pellet cores comprise a mixture of corn starch and sucrose. The mixture usually comprises 1 to 20 wt. % corn starch and 80 to 99 wt. % sucrose, in particular, about 8 wt. % corn starch and 92% sucrose.

In step (3-II) the Ruxolitinib is dissolved or suspended in a solvent. The solvent can be water, a pharmaceutically acceptable organic solvent or mixtures thereof. Preferably, the solvent is water or an alcohol. Most preferably, the solvent is methanol.

The solution or dispersion of Ruxolitinib can comprise further excipients (d). It preferably comprises a solubilizer (d1) and/or a plasticizer (d8). Generally, it is noted that all comments made above regarding the excipients (d) used in the present invention also apply for the processes of the present invention. In addition, the solution or dispersion may comprise anti-sticking agents and lubricants.

The resulting emulsion or suspension is sprayed onto the pellet core, preferably by a fluid bed dryer, e.g. Glatt GPCG 3 (Glatt GmbH, Germany).

Subsequently, the spraying step is repeated. In step (3-III) a solution or suspension comprising component (b) and optionally (c) and/or (d) is sprayed onto the pellet resulting from step (3-II). In the spraying step (3-III), preferably solubilizer (d1) and/or plasticizer (d8) are used as excipients.

Alternatively, the spraying steps (3-II) and (3-III) can be combined. In such a case, the solution or dispersion of Ruxolitinib further comprises components (b) and optionally (c) and/or excipients (d).

In a preferred embodiment, the spraying conditions are chosen such that the resulting coated spheroids have a mean particle size (D50) of 10 to 1000 μm, more preferably of 50 to 800 μm, further more preferably of 100 to 750 μm, most preferably of 250 to 650 μm.

The coated spheroids of the present invention (i.e. the primary pharmaceutical composition) may be used to prepare suitable solid oral dosage forms with modified released properties. That means, the primary pharmaceutical composition can be further processed to give a “final pharmaceutical composition”, i.e. to give a final oral dosage form.

Hence, the present invention encompasses a process for producing oral dosage forms comprising a pharmaceutical composition as received by the above-described pellet layering process, comprising the steps of
(3-V-i) optionally mixing the granulates as received by the above-described pellet layering process with further excipients,
(3-V-ii) further processing the resulting mixture into a final oral dosage form.

Preferably, step (ii) comprises
(3-V-ii-α) filling the resulting mixture into capsules,
(3-V-ii-β) filling the resulting mixture into sachets, or
(3-V-ii-γ) compressing the resulting mixture into tablets. The tablets can be film-coated (e), as described above.

Generally, it is noted that all comments made above with respect to the tablets of the present invention also apply for the process of manufacturing such a tablet and the use of the tablet of the present invention.

Consequently, further subjects of the present invention are tablets obtainable by any of the processes as described above.

All explanations above given for the process of the present invention also apply for the tablet of the present invention.

The release profile of a non-coated tablet or a coated tablet, wherein the coating is free of drug, usually shows a constant release as determined by method USP (paddle). Preferably, the slope of the initial drug release is less than 0.6 to 0.8% per minute.

In a preferred embodiment, the oral dosage form of the present invention is suitable for an administration once or twice per day, most preferably once per day. Alternatively, the oral dosage form of the present invention can be administered every second day, thrice a week, twice a week or once a week.

Finally, the present invention provides the use of the modified release tablet of the present invention as immunosuppressive agent for organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, leukemia.

The present invention is illustrated by the following examples.

### EXAMPLES

The following commercially available compounds are used in the examples below:

| | |
|---|---|
| Eudragit ® | anionic copolymer of methacrylic acid and acrylic |
| L100-55 (Röhm): | acid ethylester |
| Eudragit ® | copolymer of acrylic and methacrylic acid esters |
| RL PO (Röhm): | containing quaternary ammonium groups |
| Klucel EF | hydroxypropyl cellulose with a molecular weight of |
| | approximately 80,000 g/mol |
| Klucel HF | hydroxypropyl cellulose with a molecular weight of |
| | approximately 1,150,000 g/mol |
| Galen IQ | hydrogenated isomaltulose |
| StarLac | coprocessed lactose and starch |
| Prosolv | silicified microcrystalline cellulose |
| Granulac | lactose monohydrate |
| PVP25 | polyvinyl-2-pyrrolidinone with a molecular |
| | weight of approximately 30,000 g/mol |
| PVP90 | polyvinyl-2-pyrrolidinone with a molecular |
| | weight of approximately 1,000,000 g/mol |
| RetaLac | 50% lactose monohydrate + 50% hydroxypropyl |
| | methyl cellulose |
| Prosolv SMCC | microcrystalline cellulose + 2% silicon dioxide |
| Kollicoat ® MAE | methacrylic acid copolymer |
| 100P (BASF): | |
| Kollidon ® SR | mixture of 80% hydrophobic polyvinyl acetate, 19% |
| (BASF): | hydrophilic polyvinyl pyrrolidone, 0.8% sodium |
| | lauryl sulfate and 0.2% colloidal silicate |
| Aerosil ® 200 | highly dispersed silicium dioxide |
| (Degussa): | |
| Avicel ® PH102 | microcrystalline cellulose, with D50 particle size |
| (FMC): | of about 100 μm |
| Lubritab ® | hydrogenated vegetable oil |
| Opadry ® | film-coating |

### Example 1

### Matrix Tablet, Direct Compression

| Ruxolitinib phosphate | 50 mg (based on the free base) (17.0%) | |
|---|---|---|
| Eudragit ® L100-55 | 120 | mg (40.8%) |
| Lactose monohydrate | 60 | mg (20.4%) |
| Dicalcium phosphate anhydrate | 60 | mg (20.4%) |
| Aerosil ® 200 | 2 | mg (0.7%) |
| Magnesium stearate | 2 | mg (0.7%) |

All ingredients except magnesium stearate is blended in a free fall mixer for 15 min. Then, sieved magnesium stearate is added and the mixture is blended for further 5 min. The final blend is compressed into tablets.

### Example 2

### Matrix Tablet, Wet Granulation

| | |
|---|---|
| Ruxolitinib free base | 50 mg (based on the free base) (19.2%) |
| Kollicoat ® MAE 100P | 120 mg (46.2%) |
| Lactose monohydrate | 50 mg (19.2%) |
| Avicel ® PH102 | 34 mg (13.1%) |
| Aerosil ® 200 | 4 mg (1.5%) |
| Magnesium stearate | 2 mg (0.8%) |

Ruxolitinib, Kollicoat® and lactose are sieved (1.25 mm mesh) into the pot of a Diosna® P1-6 wet granulator and blended for 2 min. This pre-mixture is granulated, adding a suitable amount of water to gain a mixture having a “snow ball” consistency. The wet granulate is sieved (2 mm mesh) and dried for 2 h at 40° C. in a cabinet drier. The dried granulate is sieved (1.25 mm mesh) and Avicel® and Aerosil® (both sieved with 1.25 mm mesh) are added and the resulting mixture is blended for further 15 min in a free fall mixer. Sieved (500 μm mesh) magnesium stearate is added and the resulting mixture is blended in a free fall mixer for 5 min. The final blend is compressed into tablets.

### Example 3

### Dry Granulation

| | | |
|---|---|---|
| Ruxolitinib free base | 50 | mg (17.0%) |
| Eudragit L 100-55 | 120 | mg (40.8%) |
| GalenIQ 800 | 60 | mg (20.4%) |
| Dicalcium phosphate anhydrate | 60 | mg (20.4%) |
| Aerosil 200 | 2 | mg (0.7%) |
| Magnesium stearate | 2 | mg (0.7%) |

All ingredients except the Aerosil and the magnesium stearate are passed through a sieve (1 mm) and then mixed in a free fall mixer for 15 min. The pre-mix is then compacted and the resulting mixture is passed through a sieve (1 mm). Aerosil is added through a sieve (1 mm). the mixture is mix in a free fall mixer for 10 min. Magnesium stearate is added through a sieve (0.5 mm) and the mixture is mixed for another 5 min. Finally, the mixture is pressed to obtain tablets.

### Example 4

### Direct Compression

| | |
|---|---|
| Ruxolitinib phosphate | 50 mg (based on the free base) (17.0%) |
| Kollidon ® SR | 120 mg (40.8%) |
| Lactose monohydrate | 60 mg (20.4%) |
| Dicalcium phosphate | 60 mg (20.4%) |
| anhydrate | |
| Aerosil ® 200 | 2 mg (0.7%) |
| Magnesium stearate | 2 mg (0.7%) |

All ingredients, except magnesium stearate, are sieved (1 mm mesh) and blended in a free fall mixer for 15 min. Then, sieved (500 μm) magnesium stearate is added and the mixture blended for further 5 min. The final blend is compressed into tablets.

### Example 5

### Wet Granulation

### Tablet Formulation 5:

| | |
|---|---|
| Ruxolitinib phosphate | 50 mg (based on the free base) (20.0%) |
| Eudragit ® RL PO | 110 mg (44.0%) |
| Lactose monohydrate | 50 mg (20.0%) |
| Avicel ® PH102 | 34 mg (13.6%) |
| Aerosil ® 200 | 4 mg (1.6%) |
| Magnesium stearate | 2 mg (0.8%) |

Ruxolitinib, Eudragit® and lactose are sieved (1.25 mm mesh) into the pot of a Diosna® P1-6 wet granulator and blended for 2 min. This pre-mixture is granulated, adding a suitable amount of water to gain a mixture having a “snow ball” consistency. The wet granulate is sieved (2 mm mesh) and dried for 2 h at 40° C. in a cabinet drier. The dried granulate is sieved (1.25 mm mesh) and Avicel® and Aerosil® (both sieved with 1.25 mm mesh) are added and the resulting mixture is blended for further 15 min in a free fall mixer. Sieved (500 μm mesh) magnesium stearate is added and the resulting mixture is blended in a free fall mixer for 5 min. The final blend is compressed into tablets.

### Example 6

### Coated Tablet

### Tablet Core

| | |
|---|---|
| Ruxolitinib free base | 50 mg (13.8%) |
| StarLac | 200 mg (55.2%) |
| Dicalcium phosphate anhydrate | 30 mg (8.3%) |
| Aerosil 200 | 2 mg (0.6%) |
| Magnesium stearate | 2 mg (0.6%) |

All excipients, excluding magnesium stearate, are sieved (800 μm) and mixed for 15 min in a free fall mixer. Sieved (500 μm mesh) magnesium stearate is added and the resulting mixture is blended in a free fall mixer for 5 min. The final blend is compressed into tablets.

### Tablet Coating

| | |
|---|---|
| Ethyl cellulose | 60 mg (16.6%) |
| PEG 6000 | 3 mg (0.8%) |
| TEC | 15 mg (4.1%) |

The coating process is carried out on a pan coater, e.g. on a Lödige LHC 25 (Lödige GmbH, Germany). The spray pressure usually ranges from 1-1.5 bar. The product temperature varies according to the applied polymer from 32-38° C.

### Example 7

### Matrix Tablet, Wet Granulation

| | | |
|---|---|---|
| Ruxolitinib phosphate | 30 | mg (5.9%) based on free base |
| MCC, Prosolv SMCC | 66 | mg (13.0%) |
| PVP 25/90 | 12.5/44.8 | mg (11.3%) |
| Hyprolose, Klucel HF | 250 | mg (49.2%) |
| Granulac | 101.4 | mg (20.0%) |
| Magnesium stearate | 3.0 | mg (0.6%) |
| Water | | |

PVP 25 is dissolved in water. All other components, except Magnesium stearate are mixed in a high share mixer for 15 minutes. This powder is sieved through a sieve of 800 μm.

After sieving, the powder is granulated with the solution of PVP in water.

The granules are dried for 2 hours at 40° C. and sieved afterwards through a sieve of 1 mm. Magnesium stearate is added, mixed for another 5 minutes and compressed into tablets.

### Example 8

### Matrix Tablet, Wet Granulation

| | |
|---|---|
| Ruxolitinib phosphate | 30 mg (5.6%) based on free base |
| MCC, Prosolv SMCC | 66.0 mg (12.2%) |
| Klucel EF | 125 mg (23.1%) |
| Hyprolose, Klucel HF | 250 mg (46.3%) |
| Granulac | 66.4 mg (12.3%) |
| Magnesium stearate | 3.0 mg (0.5%) |
| Water | |

Klucel EF is dissolved in water. All other components, except Magnesium stearate are mixed in a high share mixer for 15 minutes. This powder is sieved through a sieve of 800 μm.

After sieving, the powder is granulated with the solution of Klucel EF in water.

The granules are dried for 2 hours at 40° C. and sieved afterwards through a sieve of 1 mm. Magnesium stearate is added, mixed for another 5 minutes and compressed into tablets.

### Examples 9-11

| | | |
|---|---|---|
| Ruxolitinib phosphate | 39.98 | mg (7.26%) |
| Retalac | 251.00 | mg (45.55%) |
| Granulac/Prosolv SMCC | 257.00 | mg (46.64%) |
| Mg stearate | 3.00 | mg (0.54%) |
| Ruxolitinib phosphate | 39.98 | mg (7.26%) |
| Retalac | 200.00 | mg (36.30%) |
| Granulac/Prosolv SMCC | 308.00 | mg (55.90%) |
| Mg stearate | 3.00 | mg (0.54%) |
| Ruxolitinib phosphate | 39.98 | mg (7.26%) |
| Retalac | 300.00 | mg (54.45%) |
| Granulac/Prosolv SMCC | 208.00 | mg (37.75%) |
| Mg stearate | 3.00 | mg (0.54%) |

Ruxolitinib, RetaLac, Granulac and Prosolv SMCC were blended in das mixer (Turbula TB10) for 15 minutes at 23 rpm.

The mixture was sieved through an appropriate sieve with 630-1000□m mesh.

Then magnesium stearate was added and again mixed for 3 minutes. The final blend was compressed into tablets with an Kirsch EK0 excenter press.

### Example 12

### Osmotic-Controlled Tablet

### Tablet Core

| | | |
|---|---|---|
| Ruxolitinib phosphate | 50 | mg (based on the free base) |
| PolyOx WSR-N80 (Dow) | 238 | mg |
| Xylitol (trade name XYLITAB 200) | 118 | mg |
| Magnesium stearate | 2 × 2 | mg |

Polyox and xylitol are combined and blended in a free fall mixer. The blended material is passed through a sieve (800 μm). The resulting material is added to a blender and the Ruxolitinib phosphate is added and the resulting mixture is mixed for 15 min. Magnesium stearate (2 mg) is added and the resulting blend is mixed for another 5 min. The blend is roller compacted. The resulting granules are transferred to a free fall mixer. Magnesium stearate (2 mg) is added and the final blend is mixed for another 15 min.

| | |
|---|---|
| PEO WSR Coagulant (Dow) | 129 mg |
| Avicel PH 200 (FMC) | 51.6 mg |
| Sodium chloride | 17.2 mg |
| FD&C #2 Blue Lake | 0.6 mg |
| Magnesium stearate | 1 mg |

Coagulant, Avicel, sodium chloride and FD&C are mixed in a free fall mixer for 15 min. Magnesium stearate is added are the final blend for the swellable layer is mixed for 15 min.

Tablet cores are formed by compressing 600 mg (400 mg Ruxolitinib-containing layer; 200 mg swellable layer) using a rotary tri-layer press (e.g. Elizabeth-HATA AP-55). Feed hopper #1 is filled with the Ruxolitinib-containing layer, feed hopper #2 is empty and feed hopper #3 is filled with the swellable layer. A tamp force of 50-65 kg is used for the Ruxolitinib-containing layer and the tamp force of 500-600 kg is used after hopper #3 and the final compression force is approx. 14 kN resulting in tablets of approx. 15 kP hardness.

### Coating

| | |
|---|---|
| Polyethylene glycol | 8.0 mg |
| Water | 40 mg |
| Acetone | 920 mg |
| Cellulose acetate | 32 mg |

Polyethylene glycol (PEG 3350) is dissolved in water and acetone and added to the. The cellulose acetate (CA 398-10 from Eastman Fine Chemical) is added to the solution and the resulting solution is mixed until homogeneous. The coating solution is applied to the tablet cores using a pan coater, e.g. on a Lödige LHC 25 (Lödige GmbH, Germany). The spray pressure usually ranges from 1-1.5 bar. The product temperature varies according to the applied polymer from 32-38° C. The so-coated tablets are dried in a convection oven. One 1200 μm diameter hole is then laser-drilled in the coating on the drug-containing composition side of the tablet to providing one delivery port per tablet.

### Example 13

### MUPS

| | |
|---|---|
| Ruxolitinib, free base micronized | 50 mg |
| Polyoxyethylenepropylene copolymer | 12 mg |
| Ethylcellulose: | 50 mg |
| PEG 4000 | 8 mg |
| Nonpareils | 80 mg |
| MCC | 400 mg |
| Polyvinylpyrrolidone | 20 mg |
| Lubritab ® | 10 mg |
| Aerosil ® | 4 mg |
| Opadry ® | 5 mg |

Ruxolitinib is suspended together with ethyl cellulose in an aqueous solution of polyoxyethylene propylene copolymer and PEG. The placebo pellets are pre-heated to 38° C. in a fluid bed dryer. Subsequently the pellets are coated with the suspension, using the following parameter:

| | | |
|---|---|---|
| Inlet temperature: | 40-80° | C. |
| Product temperature: | 35-40° | C. |
| Spray nozzle: | 1-2 | mm |
| Spray pressure: | 1-2 | bar |

After sintering at elevated temperature the pellets are blended with MCC and Aerosil® and polyvinylpyrrolidone for 25 min in a tumble blender. Afterwards, Lubritab® is added and the blend is mixed for additional 3 minutes.

The final blend is compressed on a Fette® 102 rotary press, characterized by following parameters:

| | |
|---|---|
| Hardness: | 80-110 N |
| Friability: | less than 1%. |

The tablets are film-coated in order to achieve a better compliance with an aqueous solution of Opadry® (Colorcon®):

| | | |
|---|---|---|
| Product temperature: | 37-40° | C. |
| Supply air temperature: | 40-80° | C. |
| Nozzle diameter: | 1.2 | mm |
| Spray pressure: | 1-3 | bar |

Afterwards, the tablets are sintered at 60° C. for 0.5 hour.

1. Oral dosage form for modified release comprising
(a) Ruxolitinib, and
(b) a non-erodible material.

2. Oral dosage form according to claim 1, wherein Ruxolitinib is contained in an amount of 1 to 60 weight percent, based upon the total weight of the oral dosage form.

3. Oral dosage form according to claim 1, wherein the non-erodible material has a solubility in water at 25° C. at a pH of 5.0 of less than 33 g/l.

4. Oral dosage form according to claim 1, wherein the non-erodible material has a solubility in water at 25° C. at a pH of 7.0 of more than 33 g/l.

5. Oral dosage form according to claim 1, wherein the non-erodible material is a non-erodible polymer having a weight average molecular weight from 30,000 to 3,000,000 g/mol.

6. Oral dosage form according to claim 1, wherein the non-erodible material is contained in an amount of 5 to 80 weight percent based upon the total weight of the oral dosage form.

7. Oral dosage form according to claim 1, further comprising a pore-forming material (c).

8. Oral dosage form according to claim 7, wherein the pore-forming material has a solubility in water at 25° C. and at a pH of 5.0 of more than 50 g/l.

9. Oral dosage form according to claim 7, wherein the pore-forming material is contained in an amount of 1 to 50 weight percent, based upon the total weight of the oral dosage form.

10. Oral dosage form according to claim 1, further comprising at least one further excipient (d) selected from fillers, lubricants, disintegrants, glidants, anti-sticking agents, plasticizers and mixtures thereof.

11. Oral dosage form according to claim 1 in form of a matrix tablet.

12. Oral dosage form according to claim 1 in form of a tablet comprising a core and a shell, wherein the core comprises components (a) and optionally (c) and/or (d) and wherein the shell comprises components (b) and optionally (c) and/or (d).

13. Oral dosage form according to claim 1 in form of a multiple unit pellet system.

14. Process for manufacturing the oral dosage form according to claim 10 comprising the steps of:
(1-I) providing components (a), (b), optionally (c), and optionally (d),
(1-II) optionally agglomerating the components of step (I) to yield granules,
(1-III) compressing the mixture resulting from step (I) or (II) into tablets; and (1-IV) optionally film-coating the tablets.

15. Process for manufacturing the oral dosage form according to claim 11 comprising the steps of:
(2-I) mixing components (a) and optionally (c) and/or (d),
(2-II) optionally agglomerating the components of step (I) to yield granules,
(2-III) compressing the mixture into tablets, and
(2-IV) coating the tablets with a coating comprising components (b) and optionally (c) and/or (d),

16. Process for manufacturing an oral dosage form according to claim 12 comprising the steps of:
(3-I) providing a pellet core,
(3-II) spraying a solution or suspension comprising component (a) and optionally (d) onto the pellet core,
(3-III) spraying a solution or suspension comprising component (b) and optionally (c) and/or (d) onto the pellet,
(3-IV) optionally blending the pellets with components (b) and (c) and/or (d); and
(3-V) further processing the resulting mixture into a final oral dosage form.
